(19) United States
(12) Patent Application Publication
(10) Pub. No.: US 2025/0114545 A1
(43) Pub. Date: Apr. 10, 2025

(54) Device and system for generating a continuous positive airway pressure, in particular for respiratory support in premature and newborn infants

(71) Applicant: pfm medical gmbh, Koeln (DE)

(72) Inventors: Pia Welfers, Wegberg (DE); Bianca Haase, Tuebingen (DE); Georg Kox, Wegberg (DE); Daniel Rauch, Essen (DE)

(21) Appl. No.: 18/908,510

(22) Filed: Oct. 7, 2024

(30) Foreign Application Priority Data
Oct. 9, 2023 (IT) 102023000020877

(51) Int. Cl. A61M 16/00; A61M 16/06; A61M 16/08; A61M 39/00; A61M 39/12

(52) U.S. Cl. CPC A61M 16/0063; A61M 16/0003; A61M 16/0666; A61M 16/0816; A61M 39/12; A61M 2039/0009; A61M 2202/0007; A61M 2202/02; A61M 2205/10; A61M 2205/3331; A61M 2240/00

(57) ABSTRACT

Disclosed is a system for generating a continuous positive airway pressure, in particular for respiratory support in premature and newborn infants. The system includes a connecting piece having a hollow cylinder with open ends and an inlet channel on an inlet side and a collecting channel an outlet side with the inlet cannel and collecting channel positioned opposite and axial to each other.

### BACKGROUND

The invention relates to a device and a system for generating a continuous positive airway pressure, also referred to as a CPAP (Continuous Positive Airway Pressure) device. The CPAP device is designed, for example, as a nasal CPAP device (nCPAP). CPAP devices are also used in particular for respiratory support of premature and newborn babies in the case of surfactant deficiency associated with prematurity or transient tachypnoea of the newborn.

The functional principle of CPAP devices is based on the patient breathing against a pressure that is higher than atmospheric pressure. By generating PEEP (positive end-expiratory pressure) in the alveoli through a CPAP device, an attempt is made to prevent them from collapsing in order to prevent atelectasis and thus a reduction in functional residual capacity.

In contrast, normal respiratory devices only provide an air supply during inhalation, for example, and a negative pressure is generated during exhalation. For example, DE 31 19 814 A1 discloses a respiratory device with a controllable breathing gas source and a device for generating negative pressure, which supplies the patient with high-frequency high-pressure gas pulses during the inspiratory phase. The respiratory device supplies at least one jet nozzle in a tracheal tube with the high-pressure gas pulses via the controllable breathing gas source. To improve the effectiveness in the expiratory phase and in particular the elimination of CO₂ while at the same time avoiding obstruction of spontaneous breathing processes, such as coughing bursts, it is provided that the tracheal tube is connected, at least in the expiratory phase, to a device for generating negative pressure, which establishes a connection between the proximal end of the tube and the ambient atmosphere in both breathing phases. The respiratory device can be used for patients with respiratory disorders, but the negative pressure generated in the expiratory phase does not prevent the alveoli from collapsing in premature and newborn babies. Other comparable ventilators are known, for example, from U.S. Pat. Nos. 3,977,432 A or 3,794,072 A.

EP 0 447 443 A1 discloses a device for generating a continuous positive airway pressure (CPAP) by means of an ejection process, comprising a breathing channel which opens to the atmosphere at its free end and can be provided at its other end with a coupling piece for attachment to the nose and/or mouth of the patient, and an inlet channel for fresh gas which is connected to the breathing channel at a point between its ends and the flow of which is adjustable in order to achieve an adjustable positive pressure in the breathing channel. The breathing channel has a first branch channel which can be connected to the coupling piece and a second branch channel which is open to the atmosphere, the two branch channels forming an angle with each other. The inlet channel is located essentially in the extension of the first branch channel and is connected to the second branch channel in such a way that the fresh gas flow is directed mainly coaxially into the first branch channel, thereby causing an ejection process. The cross-sectional area of the respective branch channel is many times larger than the smallest cross-sectional area of the inlet channel. The length of each branch channel is relatively short and is preferably five times its internal diameter. The breathing channel is assembled with the inlet channel to form a compact unit which can be attached to the nose and/or mouth of the patient by means of an adhesive strip or equivalent means.

WO 99/24101 A1 criticizes the complicated design of the two CPAP and nCPAP devices mentioned above, in particular the separate inlet and outlet channels, which means that the devices cannot be easily sterilized and are expensive disposable items. Furthermore, the devices are not flexible in handling and in connection with compressed air generation devices and pressure measuring devices. According to WO 99/24101 A1, these disadvantages are avoided by a device for generating a continuous positive airway pressure (CPAP device), in particular a nasal CPAP device (nCPAP device), with a hollow body in which an overpressure can be built up; a first opening provided in a side wall of the hollow body for supplying an air flow directed into the hollow body and for discharging the exhaled air flow; a connecting piece attachable to the hollow body for connecting the hollow body to a nasal and/or mouth attachment; and a spacer attachable to the hollow body, to which a flow nozzle for directing the air flow onto the opening can be attached.

P 1 897 577 A1 discloses a further development of the device from WO 99/24101 A1 with an additional inlet for a flow of medication.

EP 0 658 356 A1 further discloses a CPAP device comprising a pair of nasal attachment pieces each with a cannula tip for insertion into the nostrils of a patient.

GB 2570052 B discloses a CPAP device with multiple sealable ports to provide a variety of different ventilation therapies.

EP 2 269 676 A2 discloses a CPAP device with a controllable valve in the compressed air supply in order to adjust the supplied air flow.

When providing respiratory support for premature and newborn babies using a CPAP device, it is particularly important to ensure that the load on the head of the premature and newborn baby is as low as possible, as the skull bones are still soft and malleable in the first few weeks after birth. Continuous stress on the skull caused by the CPAP device can lead to pressure points and deformations, but this should be avoided.

The invention is therefore based on the task of providing a CPAP device which is optimized for use in premature and newborn infants and, in particular, minimizes the load on the skull of the premature and newborn infant.

The problem is solved in accordance with the invention by a device for generating a continuous positive airway pressure, in particular for respiratory support in premature and newborn babies, comprising:
an inlet channel on the inlet side, for connection to a compressed air supply,
a collecting channel on the outlet side for connection to a respiratory mask or a mononasal pharyngeal tube or a binasal prong,
a connecting piece between the inlet channel on the inlet side and the collecting channel on the outlet side, the connecting piece having at least three openings to the environment,
which is characterized in that
the connecting piece is designed as a hollow cylinder with open ends and the inlet channel on the inlet side and the collecting channel on the outlet side are arranged opposite each other in the hollow cylinder wall of the connecting piece and axially to each other.

A compressed air/oxygen flow is introduced into the device according to the invention via the inlet channel on the inlet side, whereby the compressed air/oxygen flow introduced has an increased pressure compared to the ambient atmosphere. The compressed air/oxygen flow is condensed by the inner diameter of the inflow channel. The resulting increased flow velocity (Venturi effect) leads to a pressure drop (Bernoulli effect) when the air exits the inlet channel into the connecting piece and during the transition into the axially arranged collecting channel, which can also draw in ambient air at high air flows. The device according to the invention thus supports the oxygen supply during inspiration and, as in the prior art, ensures improved gas exchange due to the excess pressure generated.

The connecting piece has at least three, preferably four, openings to the surroundings. The openings are used to allow breathing air to escape during expiration. The open ends of the hollow cylindrical connecting piece form two of the three openings and the third opening is conveniently arranged in the hollow cylinder wall of the connecting piece. This ensures that the breathing air can always escape and that accidental closure of all three openings of the connecting piece by a user with just one hand can be virtually ruled out. This safety function prevents the lungs of premature and newborn babies from being overstretched due to excessive pressure and prevents alveoli from bursting (pneumothorax).

The device according to the invention has a minimalistic design with only an inlet channel on the inlet side, a collection channel on the outlet side and a connecting piece between the two channels. The hollow cylindrical connecting piece automatically forms two of the three openings through the two open ends. This allows the device to be particularly compact and lightweight, so that stress on the skull of a premature and newborn baby is significantly reduced. Furthermore, the axial alignment of the inlet channel on the inlet side and the collecting channel on the outlet side optimizes the flow within the connecting piece, which reduces the overall size and consequently the overall weight of the device.

A hollow cylindrical connector optimizes the compressed air/oxygen flow within the connector, both during inspiration and expiration. The curved inner wall of the hollow cylindrical connector directs the compressed air/oxygen flow better from the flow channel into the collection channel. The curved inner surface of the hollow cylindrical connecting piece ensures that the air flow entering the connecting piece through the inlet channel on the inlet side is directed into the collection channel on the outlet side.

In principle, the device according to the invention can be used or combined with a wide variety of devices for respiratory support, whereby the aforementioned respiratory mask, the aforementioned mononasal tube or the aforementioned binasal prong are the most common devices for respiratory support.

In contrast to the prior art according to WO 99/24101 A1 and EP 1 897 577 A1, the device according to the invention does not require the hollow body in which the positive pressure is generated according to the prior art. This significantly reduces the weight of the device according to the invention compared to this prior art.

The device according to EP 0 658 356 A1 is fixed directly to the patient's head and is therefore only suitable for use with premature and newborn babies to a limited extent.

In an advantageous variant of the invention, the inlet channel on the inlet side and the collecting channel on the outlet side are straight. The device according to the invention thus has an axial flow channel from the inlet of the inflow channel via the connecting piece to the outlet of the collecting channel. This is not only beneficial for the flow behavior within the device according to the invention, but also enables a flexible positioning of the connection hose at the inlet-side inflow channel. In the prior art according to WO 99/24101 A1, EP 1 897 577 A1, EP 0 658 356 A1 and WO 90/24101 A1, the compressed air connection on the inlet side is angled towards the respiratory connection on the outlet side, which significantly restricts the positioning of both the device and the hose connected to the compressed air supply on the inlet side. However, especially in the case of premature and newborn babies, it is essential that the device is positioned in such a way that unnecessary strain on the patient's head is effectively avoided. The axial design of the device according to the invention facilitates positioning, as the compressed air hose connected on the inlet side in particular can be positioned much more freely.

According to an expedient variant of the invention, the device comprises a connector on collecting channel of the outlet-side, in particular a female M15 adapter. The adapter is designed, for example, in accordance with DIN EN ISO 80369-2. The adapter allows the device according to the invention to be connected quickly, easily and securely to a respiratory mask or a mononasal pharyngeal tube or a binasal prong, which for example have a corresponding mating connector, such as a male M15 adapter.

According to a variant according to the invention, the connector comprises ribs on its outer surface as reinforcement and/or grooves as a gripping element. The reinforcing ribs ensure easy connection, as the connector does not deform during handling, which could, for example, make it difficult to insert an M15 male adapter into an M15 female adapter. The grooves serving as a gripping element generally improve the handling of the device.

In an advantageous variant of the invention, the ribs extend from the connecting piece over the outer surface of the outlet-side collecting channel and the outer surface of the connector. Thus, in addition to the connector, the outer surface of the collecting channel is also stabilized and protected against deformation. A negative influence of deformations on the patient-side (outlet-side) flow in the collecting channel is thus prevented.

According to a particularly preferred variant of the invention, the device is designed in one piece. The one-piece design of the device according to the invention allows the flow properties to be optimized. In particular, dead spaces in the area of connections, such as a detachable connection between the outlet-side collecting channel and optional connector, are avoided. Dead spaces of this kind can, for example, make it difficult to exhale CO₂-containing breathing air and are difficult to sterilize using conventional methods and promote the colonization of germs during use. Thanks to the one-piece design, such dead spaces are avoided or cannot arise due to unsafe or imprecise connection points.

According to a variant of the invention, the device is made of plastic. This makes the device easy and inexpensive to manufacture. Furthermore, the weight of the device can be minimized, which makes the device particularly suitable for respiratory support in premature and newborn babies. For example, a device according to the invention including the optional connector in a one-piece plastic design has a weight of less than 5 g, in particular less than 4 g.

In an expedient variant of the invention, the inlet channel on the inlet side has a smaller internal diameter than the collecting channel on the outlet side. Due to the smaller internal diameter of the inlet channel, the compressed air/oxygen flow is more condensed. The resulting increased flow velocity (Venturi effect) leads to an optimum pressure drop (Bernoulli effect) when the air exits the inlet channel into the connecting piece and during the transition into the axially arranged larger collecting channel, which can also draw in ambient air at high air flows.

According to a practical variant, the inlet channel on the inlet side has a diameter of 2 mm to 4 mm, in particular 3 mm.

According to a further variant according to the invention, the outlet-side collecting channel has a diameter of 3 mm to 5 mm, in particular 4 mm.

In an advantageous variant of the invention, the inlet channel on the inlet side protrudes into the connecting piece and the collecting channel on the outlet side ends at the wall of the connecting piece. In particular, this optimizes the transition from the inlet-side inlet channel to the outlet-side collecting channel in the connecting piece. The end of the inlet channel projecting into the connecting piece is positioned in such a way that a sufficient flow of compressed air/oxygen flows into the outlet-side collecting channel at a higher pressure than the surrounding atmosphere. The end of the outlet-side collecting channel arranged on the connecting piece ends at the curved wall of the hollow cylindrical connecting piece, whereby the curved wall of the hollow cylindrical connecting piece directs the compressed air/oxygen flow from the flow channel into the collecting channel.

According to an appropriate variant of the invention, the distance between the inlet channel on the inlet side and the collecting channel on the outlet side in the area of the connecting piece is between 3.5 mm and 5.5 mm, in particular 4.5 mm. In particular, the dimensions of the connecting piece, the inlet-side inlet channel and the outlet-side collecting channel are designed such that a positive end-expiratory pressure (PEEP) of at least 3 cm H₂O up to 12 cm H₂O is achieved, preferably between 5 cm H₂O and 8 cm H₂O.

According to an expedient variant of the invention, the open ends of the hollow cylindrical connecting piece form two of the at least three openings. The at least remaining third opening is arranged in the hollow cylindrical wall of the connecting piece.

In an advantageous variant, the third opening is arranged in the middle between the inlet channel on the inlet side and the collecting channel on the outlet side in the hollow cylinder wall. In this position, the third opening has the least influence on the air flow within the connecting piece, as the distance to both the inlet channel on the inlet side and the collecting channel on the outlet side is at a maximum.

According to a variant of the invention, the height of the hollow cylindrical connecting piece is smaller than the diameter of the hollow cylindrical connecting piece.

According to a further variant of the invention, the height of the hollow cylindrical connecting piece is greater than the outer diameter of the inlet channel on the inlet side. The circular connecting piece thus also serves as a stop for an air supply hose placed on the inlet channel and prevents the openings on the connecting piece from being closed.

The problem is further solved by a system for generating a continuous positive airway pressure, in particular for respiratory support in premature and newborn infants, comprising:
a compressed air supply that provides an air flow at a constant pressure;
a ventilator mask, a mononasal tube or a binasal prong for the premature or newborn infant; and
a device according to the invention, wherein the inflow channel on the inlet side is connected to the compressed air supply via a first air tube and the collecting channel on the outlet side is connected to the respiratory mask, the mononasal pharyngeal tube or the binasal prong via a second air tube.

In a variant according to the invention, the compressed air supply provides a constant output pressure of between 2.2 and 6.6 bar, in particular between 3.3 and 5.5 bar.

### BRIEF DESCRIPTION OF THE DRAWINGS

The invention is explained in more detail below with reference to embodiments shown in the figures. It shows:

FIG. 1a perspective view of a first embodiment of a device according to the invention for generating a continuous positive airway pressure,

FIG. 2a sectional view of a second embodiment of a device according to the invention for generating a continuous positive breathing pressure,

FIG. 3a first view of the first embodiment from FIG. 1 with an exemplary incorrect handling of the device according to the invention in the area of the connecting piece,

FIG. 4a second view of the first embodiment from FIG. 1 with an exemplary incorrect handling of the device according to the invention in the area of the connecting piece,

FIG. 5a perspective view of a third embodiment of a device according to the invention in connection with an air supply tube and a mononasal pharyngeal tube,

FIG. 6a perspective view of the device in FIG. 5 during respiratory support of a premature and newborn infant,

FIG. 7a perspective view of a fourth embodiment of a device according to the invention in connection with an air supply tube and a mononasal pharyngeal tube,

FIG. 8a perspective view of the device of FIG. 7 during respiratory support of a premature and newborn infant, and

FIG. 9a side view of a fifth embodiment of a device according to the invention.

### DETAILED DESCRIPTION

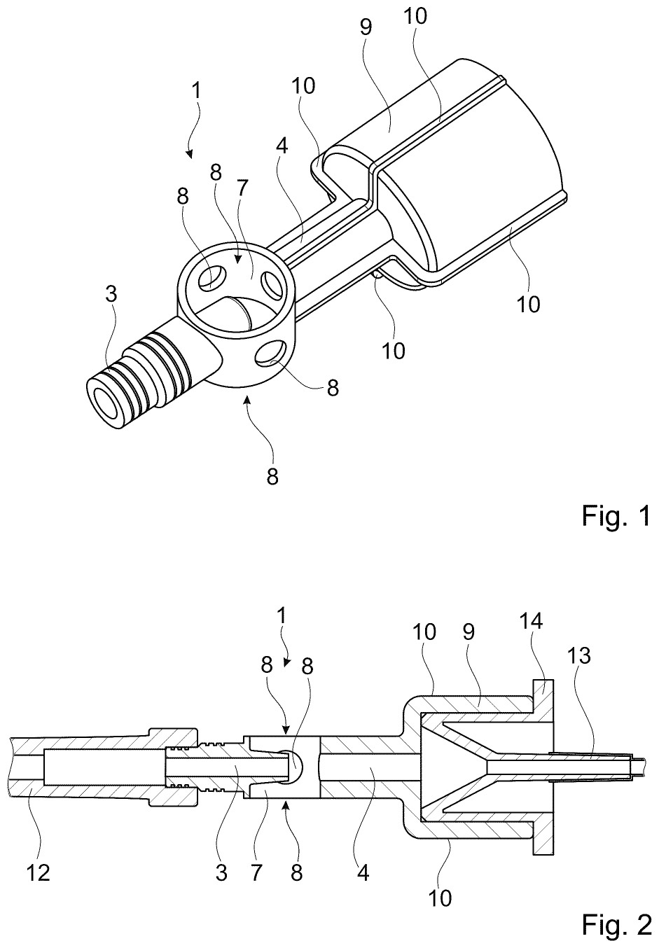

FIG. 1 shows a perspective view of a first embodiment of a device 1 according to the invention for generating a continuous positive airway pressure, in particular for respiratory support in premature and newborn infants 2. Such devices are also known as CPAP (Continuous Positive Airway Pressure).

The device 1 according to the first embodiment shown in FIG. 1 comprises an inlet channel 3 on the inlet side, a collecting channel 4 on the outlet side and a connecting piece 7 between the inlet channel 3 on the inlet side and the collecting channel 4 on the outlet side. Furthermore, the device 1 comprises a connector 9 on the collecting channel 4 on the outlet side.

According to the invention, the connecting piece 7 is designed as a hollow cylinder with open ends (hollow cylinder-shaped). The inlet channel 3 on the inlet side and the collection channel 4 on the outlet side are arranged opposite each other in the hollow cylinder wall of the connecting piece 7 and axially to each other. Furthermore, the inlet channel 3 on the inlet side and collecting channel 4 on the outlet side are straight, so that they run axially with each other.

The hollow cylindrical connecting piece 7 has at least three openings 8, whereby the connecting piece 7 according to the first embodiment in FIG. 1 has four openings 8. The exhausted air can escape into the environment via these several openings 8. The open ends of the hollow cylindrical connecting piece 7 form two of these four openings 8. The remaining two openings 8 are arranged opposite each other on the circular connecting piece 7 and are positioned centrally between the inlet channel 3 on the inlet side and the collecting channel 4 on the outlet side. Including the two open ends of the circular connecting piece 7, there are therefore four openings 8 in the connecting piece 7, which create a connection to the environment. This makes it virtually impossible for a single hand to cover all openings 8 at the same time.

The inlet channel 3 on the inlet side is used for connection to a compressed air supply (not shown), in particular a compressed air/oxygen supply. A compressed air/oxygen flow is introduced from the compressed air supply into the device 1 according to the invention via the inlet channel 3 on the inlet side, whereby the compressed air/oxygen flow introduced has an increased pressure compared to the ambient atmosphere. For example, the compressed air supply is connected to the inlet-side inlet channel 3 via an air supply hose 12, whereby the air supply hose 12 is conveniently attached to the free end of the inlet-side inlet channel 3.

The compressed air/oxygen flow is condensed by the inner diameter of the inlet channel 3. The resulting increased flow velocity (Venturi effect) leads to a pressure drop (Bernoulli effect) when the air exits the inlet channel 3 into the connecting piece 7 and during the transition into the axially arranged collecting channel 4, which can also draw in ambient air at high air flows. The device 1 according to the invention thus supports the oxygen supply during inspiration and, as in the prior art, ensures improved gas exchange due to the excess pressure generated. Furthermore, the inlet pressure at the inlet channel 3 can be reduced as a result, which significantly reduces the noise generated. A noise reduction is particularly advantageous in neonatology, as the noise generated additionally stresses the premature or newborn babies 2.

The collecting channel 4 is connected to a respiratory mask 5 or a mononasal pharyngeal tube 6 or a binasal prong. For this purpose, a connector 9, such as a female M15 adapter according to DIN EN ISO 80369-2, is preferably arranged on the collecting channel 4. The mononasal tube 6 is conveniently connected directly or via a connecting tube 13 to the collecting channel 4 of the device 1 according to the invention, just as, for example, a respiratory mask 5 can be connected directly to the connector 9 or via a connecting tube 13 to the latter. For this purpose, the connecting tube 13, the respiratory mask 5, the mononasal tube 6 or the binasal prong has, for example, a connecting counterpart 14, in particular a male M15 adapter, which can be inserted into the female M15 adapter (connector 9).

The connector 9 has a total of four reinforcing ribs 10 on its outer surface. The four reinforcing ribs 10 are evenly distributed around the circumference of the connector 9, i.e. at a distance of 90°. According to the first embodiment shown in FIG. 1, the reinforcing ribs 10 extend from the connecting piece 7 over the outer surface of the outlet-side collecting channel 4 and the outer surface of the connector 9. Thus, the entire outlet-side area of the device 1 according to the invention is reinforced.

In the embodiment shown in FIG. 1, the height of the hollow cylindrical connecting piece 7 is greater than the outer diameter of the inlet channel 3. The circular connecting piece 7 therefore also serves as a stop for the air supply hose 12 and prevents the openings 8 on the connecting piece 7 from being closed.

The device 1 according to the invention is preferably designed as a single piece and, in particular, is made of a plastic.

According to the first embodiment shown in FIG. 1, the inlet channel 3 on the inlet side has a smaller diameter than the collection channel 4 on the outlet side. For example, the inlet channel 3 on the inlet side has a diameter of 2 mm to 4 mm, in particular 3 mm, and the collecting channel 4 on the outlet side has a diameter of 3 mm to 5 mm, in particular 4 mm.

As can also be seen from FIG. 1, the inlet-side inlet channel 3 projects into the connecting piece 7 and the outlet-side collecting channel 4 ends at the wall of the connecting piece 7. The distance between the inlet-side inlet channel 3 and the outlet-side collecting channel 4 in the area of the connecting piece 7 is preferably between 3.5 mm and 5.5 mm, in particular 4.5 mm.

FIG. 2 shows a sectional view of a second embodiment of a device 1 according to the invention for generating a continuous positive airway pressure. The second embodiment shown in FIG. 2 essentially corresponds to the first embodiment in FIG. 1. In FIG. 2, the device 1 according to the invention is connected to an air supply hose 12 at the inlet-side inlet channel, which is connected to a compressed air/oxygen supply (compressed air source) at the other end. The connector 9 on the outlet-side collecting channel 4 is connected via a corresponding mating connector 14 to a connecting tube 13, which in turn merges into a mononasal tube 6 or is connected to such a tube. FIG. 2 shows in particular that the flow channel through the device 1 according to the invention is axial and has no bends.

The connector 9 and connecting counterpart 14 are not only precisely matched to each other in order to create a secure connection between the device 1 according to the invention and the mononasal pharyngeal tube 6, the respiratory mask 5 or the binasal prong, but also to minimize the dead space within the connection. An unnecessary dead space makes it more difficult to exhale CO₂, especially for premature and newborn babies 2 and should therefore be avoided or at least minimized.

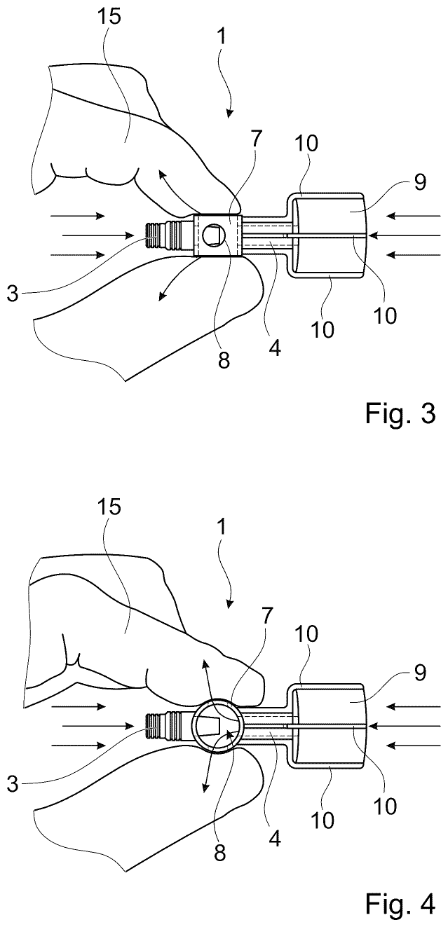

FIGS. 3 and 4 show a first and second views, respectively, of the first embodiment of FIG. 1 with an exemplary incorrect handling of the device 1 according to the invention in the area of the connecting piece 7 by the medical personnel. As shown, the hand 15 of the medical personnel grips the device 1 according to the invention in the area of the connecting piece 7 and partially covers the openings 8 to the surroundings. However, due to the number and arrangement of the openings 8, the invention prevents the medical personnel from unintentionally covering and consequently closing all openings 8 to the surroundings at the same time. If there is no longer a connection to the environment, the premature and newborn infant 2 would have to breathe against the compressed air/oxygen supply on the inlet side, which is not possible due to the fact that the lungs are not yet fully functional. This would therefore lead to overstretching of the lungs of the premature and newborn infant, which is prevented by the invention.

If the medical personnel grips the device 1 in the area of the upper and lower openings 8 of the hollow cylindrical connecting piece 7, as shown in FIG. 3, the exhaled air can escape via the openings 8 in the circumferential wall of the hollow cylinder. If the medical personnel grips the device 1 in the area of the two openings 8 in the circumferential wall of the hollow cylindrical connecting piece 7, the exhaled air can escape from the upper and lower openings 8 of the hollow cylindrical connecting piece 7 (FIG. 4).

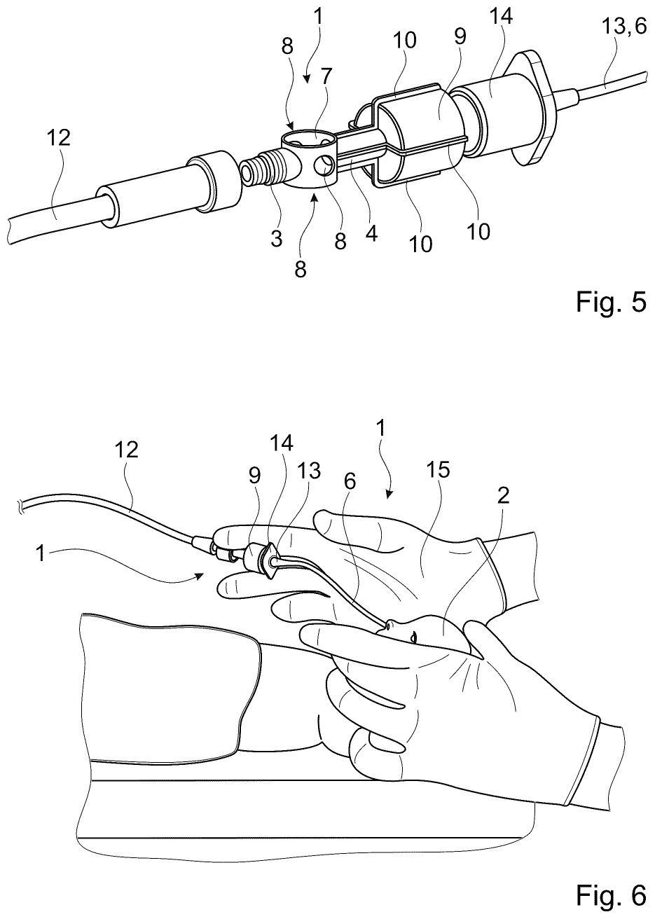

FIG. 5 shows a perspective view of a third embodiment of a device 1 according to the invention in connection with an air supply tube 12 and a mononasal tube 6. The device 1 essentially corresponds to the device in FIG. 1, which is connected to an air supply tube 12 on the inlet side. A mating connector 14 is inserted into the connector 9 on the outlet-side collecting channel 4, which is connected to a mononasal pharyngeal tube 6 via a connecting tube 13 or merges into it.

The combination of the connecting piece 7 with the respiratory mask 5, the mononasal pharyngeal tube 6 or the binasal prong and the connected compressed air supply (not shown) forms a system according to the invention for generating a continuous positive airway pressure, in particular for respiratory support in premature and newborn infants 2.

FIG. 6 shows the use of the system or device 1 of FIG. 5 during respiratory support via a mononasal pharyngeal tube of a premature and newborn infant 2. It can be seen from FIG. 6 that the straight, axial design of the device 1 according to the invention hardly restricts the guidance of the air supply tube 12, in particular in comparison with the angled devices known from the prior art.

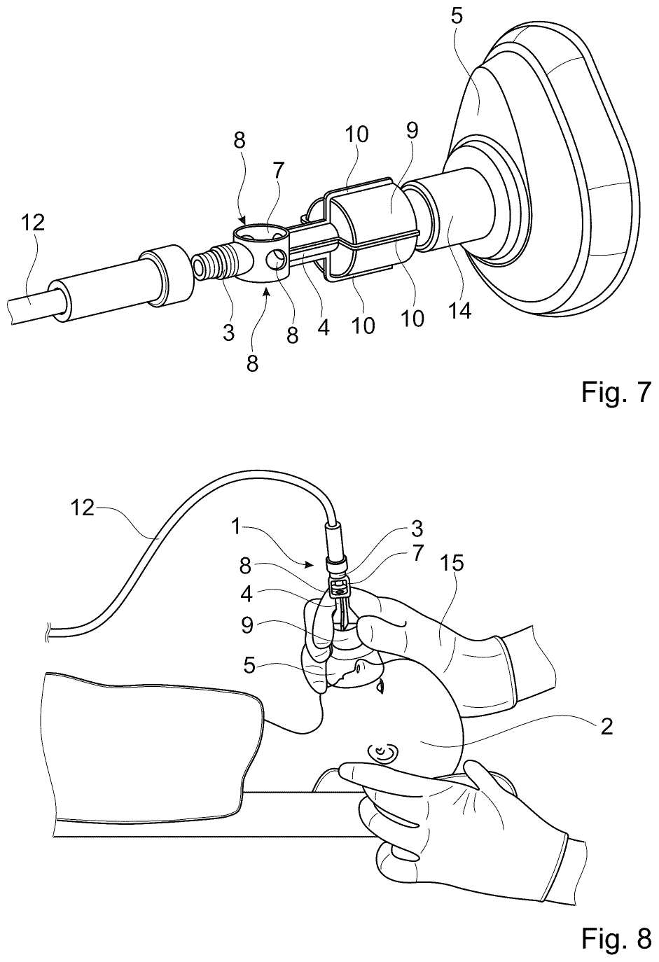

FIG. 7 shows a perspective view of a fourth embodiment of a device 1 according to the invention in connection with an air supply tube 12 and a respiratory mask 5. The device 1 essentially corresponds to the device in FIG. 1, which is connected to an air supply tube 12 on the inlet side. A mating connector 14 arranged directly on the respiratory mask 5 is inserted into the connector 9 on the outlet-side collecting channel 4.

FIG. 8 shows the use of the system or device 1 of FIG. 7 during respiratory support of a premature and newborn infant 2 by means of a respiratory mask 5. Here, too, it can be seen that the straight, axial design of the device 1 according to the invention hardly restricts the guidance of the air supply tube 12.

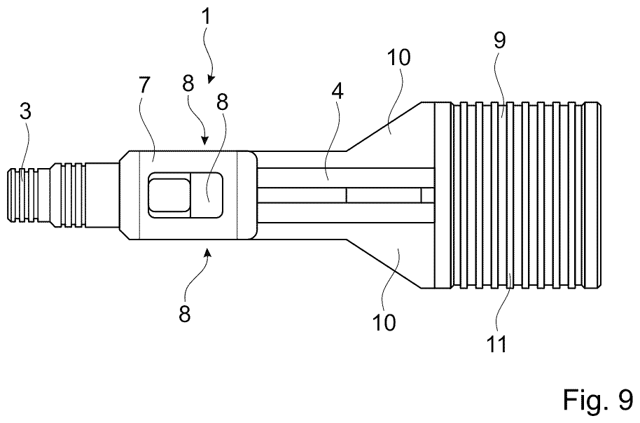

FIG. 9 shows a side view of a fifth embodiment of a device 1 according to the invention for generating a continuous positive airway pressure, in particular for respiratory support in premature and newborn infants 2. The fifth embodiment shown in FIG. 9 differs from the previous embodiments in particular in that the connector 9 additionally has grooves 11 to improve the grip. The grooves 11 are arranged on the circular outer surface of the connector 9. According to the fifth embodiment example, the reinforcing ribs 10 extend from the connecting piece 7 over the outlet-side collecting channel 4 and along the radial side surface of the connector 9.

Furthermore, in the fifth embodiment example, the shape of the openings 8 in the circular circumferential wall of the connecting piece 7 has been adapted, which are now oval/rectangular and no longer round as in the previous embodiments shown. In principle, the openings 8 can have any shape as long as they are suitable for removing the exhaled air from the device 1.

### LIST OF REFERENCE SYMBOLS

1 device
2 premature/newborn infant
3 inlet channel
4 collecting channel
5 respiratory mask
6 mononasal pharyngeal tube
7 connecting piece
8 opening (connecting piece)
9 connector
10 reinforcing rib
11 groove (grip element)
12 Air supply hose
13 connecting tube (respiratory mask/mononasal tube)
14 connecting counterpart
15 hand (medical personnel)

1. A device for generating a continuous positive airway pressure for respiratory support in premature and newborn infants, comprising:
an inlet channel on the inlet side, for connection to a compressed air supply,
a collecting channel on the outlet side for connection to a respiratory mask or a mononasal pharyngeal tube or a binasal prong,
a connecting piece between the inlet channel on the inlet side and the collecting channel on the outlet side, the connecting piece having at least three openings to the environment, wherein
the connecting piece comprises a hollow cylinder with open ends and the inlet channel on the inlet side and the collecting channel on the outlet side are arranged opposite one another in the hollow cylinder wall of the connecting piece and axially to one another.

2. A device according to claim 1, wherein the inlet channel on the inlet side and the collecting channel on the outlet side are straight.

3. A device according to claim 1, further comprising a connector at the collecting channel on the outlet side, in particular a female M15 adapter.

4. A device according to claim 3, wherein the connector comprises on its outer surface ribs as reinforcement and/or grooves as grip element.

5. A device according to claim 4, wherein the ribs extend from the connector over the outer surface of the collecting channel on the outlet side and the outer surface of the connector.

6. A device according to claim 5, wherein the device is formed in one piece.

7. A device according to claim 6, wherein the device is made of a plastic.

8. A device according to claim 7, wherein the inlet channel on the inlet side has a smaller diameter than the collecting channel on the outlet side.

9. A device according to claim 8, wherein the inlet channel on the inlet side projects into the connecting piece and the collecting channel on the outlet side ends at the wall of the connecting piece.

10. A device according to claim 9 wherein the open ends of the hollow cylindrical connector form two of the three openings and the third opening is arranged in the hollow cylindrical wall of the connector.

11. A device according to claim 10, wherein the third opening is arranged centrally between the inlet channel on the inlet side and the collecting channel on the outlet side in the hollow cylinder wall.

12. A device according to claim 11, wherein the height of the hollow cylindrical connector is smaller than the diameter of the hollow cylindrical connector.

13. A device according to claim 12, wherein the height of the hollow cylindrical connecting piece is greater than the outer diameter of the inlet channel on the inlet side.

14. A system for providing continuous positive airway pressure, particularly for respiratory support in premature and newborn infants, comprising:
a compressed air supply providing a flow of air at a constant pressure;
a respiratory mask, a mononasal pharyngeal tube or a binasal prong for the premature or newborn infant; and
a device according to claim 1, wherein the inlet channel on the inlet side is connected to the compressed air supply via a first air hose and the collection channel on the outlet side is connected to the respiratory mask, the mononasal pharyngeal tube or the binasal prong via a second air hose.

15. A system according to claim 14, wherein the compressed air supply provides a constant output pressure between 2.2 and 6.6.

16. A system according to claim 14, wherein the compressed air supply provides a constant output pressure between 3.3 and 5.5 bar.
